(19) United States
(12) Patent Application Publication
(10) Pub. No.: US 2012/0029535 A1
(43) Pub. Date: Feb. 2, 2012

(54) DEVICES AND METHODS FOR TRANSMURAL ANCHOR DELIVERY VIA A TUBULAR BODY

(76) Inventor: Paul Swain, London (GB)

(21) Appl. No.: 13/196,812

(22) Filed: Aug. 2, 2011

Related U.S. Application Data
(63) Continuation of application No. 11/123,889, filed on May 6, 2005, now abandoned.
Application No. 11/123,889 is a continuation-in-part of application No. 11/124,634, filed on May 5, 2005, now Pat. No. 8,070,743.
Application No. 11/124,634 is a continuation-in-part of application No. 11/025,364, filed on Dec. 29, 2004, now abandoned.
Application No. 11/025,364 is a continuation-in-part of application No. 10/698,148, filed on Oct. 31, 2003.
(60) Provisional application No. 60/568,929, filed on May 7, 2004.
(60) Provisional application No. 60/569,442, filed on May 7, 2004.
(60) Provisional application No. 60/613,917, filed on Sep. 27, 2004.
(60) Provisional application No. 60/534,056, filed on Dec. 31, 2003.
(60) Provisional application No. 60/569,442, filed on May 7, 2004.
(60) Provisional application No. 60/613,917, filed on Sep. 27, 2004.
(60) Provisional application No. 60/422,987, filed on Nov. 1, 2002.
(60) Provisional application No. 60/428,483, filed on Nov. 22, 2002.
(60) Provisional application No. 60/430,857, filed on Dec. 3, 2002.
(60) Provisional application No. 60/437,513, filed on Dec. 30, 2002.
(60) Provisional application No. 60/448,817, filed on Feb. 21, 2003.
(60) Provisional application No. 60/480,485, filed on Jun. 21, 2003.

(51) Int. Cl. A61B 17/04

(52) U.S. Cl. 606/144

(57) ABSTRACT

Disclosed is a transmural tissue anchor deployment system and method, for attachment to a tissue wall. The method can include the steps of advancing the tubular body through a single, non-plicated tissue wall of the gastrointestinal tract; ejecting the tissue attachment structure out of the tubular body such that the retention surface rests against a serosal surface of the tissue wall; and withdrawing the tubular body proximally across the tissue wall, wherein the tension element spans the single non-plicated tissue wall after withdrawal of the tubular body.

### CROSS-REFERENCE TO RELATED APPLICATIONS

This application claims priority under 35 U.S.C. §120 as a continuation application of U.S. patent application Ser. No. 11/123,889 filed on May 6, 2005, which in turn claims priority under 35 U.S.C. §119(e) from U.S. Provisional Application Ser. No. 60/568,929 filed May 7, 2004, the entireties of which is hereby incorporated by reference. This application also claims priority as a continuation-in-part application of U.S. patent application Ser. No. 11/124,634 filed on May 5, 2005, which claims the benefit of U.S. provisional patent application 60/569,442, filed on May 7, 2004 and U.S. provisional patent application 60/613,917, filed on Sep. 27, 2004; U.S. patent application Ser. No. 11/124,634 is also a continuation-in-part of U.S. patent application Ser. No. 11/025,364 filed on Dec. 29, 2004, which claims the benefit of U.S. Provisional App. No. 60/534,056 filed on Dec. 31, 2003, U.S. Provisional App. No. 60/569,442 filed on May 7, 2004, and U.S. Provisional App. No. 60/613,917 filed on Sep. 27, 2004; U.S. patent application Ser. No. 11/025,364 is a continuation-in-part of U.S. patent application Ser. No. 10/698,148 filed on Oct. 31, 2003, which in turn claims priority to U.S. Provisional App. No. 60/422,987 filed on Nov. 1, 2002, U.S. Provisional App. No. 60/428,483 filed on Nov. 22, 2002, U.S. Provisional App. No. 60/430,857 filed on Dec. 3, 2002, U.S. Provisional App. No. 60/437,513 filed on Dec. 30, 2002, U.S. Provisional App. No. 60/448,817 filed on Feb. 21, 2003, and U.S. Provisional App. No. 60/480,485 filed on Jun. 21, 2003. All of the foregoing applications are hereby incorporated by reference in their entireties.

### BACKGROUND OF THE INVENTION

1. Field of the Invention

The present invention relates to devices and methods for performing gastric surgery, particularly for facilitating gastric surgery using endoscopic methods, as described below.

2. Description of the Related Art

Gastrointestinal sleeve devices for treatment of obesity have been described in prior applications, as have various devices and methods for attachment of a gastrointestinal sleeve device within a patient's digestive tract. The present invention is directed to soft intragastric frames that may be used to house various devices used during surgery. The present invention is also directed to new devices and methods for sewing through an endoscope.

### SUMMARY OF THE INVENTION

A tissue anchor deployment system, for advancing through a channel in an endoscope, comprising: a tubular body, having a sharpened distal end; a tissue attachment structure within the tubular body; and a removable sheath surrounding at least the sharpened distal end, for isolating the sharpened distal end from a wall of the channel.

An intragastric support frame or implantation in the stomach, comprising: at least a first and a second inflatable balloon, each having an elongate curved body with a proximal end and a distal end, at least a first and a second inflatable balloon connected together at each of the proximal and distal ends to form a support frame; wherein the fully assembled and inflated support frame is sufficiently dimensioned to prevent passage through the pyloris.

### BRIEF DESCRIPTION OF THE DRAWINGS

FIG. 1 shows an intragastric soft building frame in a convex-outward configuration.

FIG. 2 shows an intragastric soft building frame in a concave-outward hourglass configuration.

FIGS. 3A-3B and 4A-4E show devices and methods for sewing through a conventional endoscope.

### DETAILED DESCRIPTION OF THE PREFERRED EMBODIMENT

An intragastric soft building frame can be formed as a structure made out of balloons using three or four banana-shaped balloons connected at the top and bottom into a frame. They could be assembled together inside the stomach or could be pre-assembled and expanded inside the stomach to form the necessary shape. Suitable connectors can be provided on the appropriate surfaces of the balloons for assembling the building frame together in the desired configuration. The device could be used for parking objects for use during an operation, for example an endoscopic camera, surgical instruments or components, or implantable devices. Alternatively, it could be implanted inside the stomach for short or extended periods of time and could support other structures, which could process or conduct fluid or solid materials through the stomach. It could hold a camera for long-term use. The device provides a light, soft structure. The device could be inflated with a gas, such as air or helium, or with a liquid, such as saline solution. Mucosal contact points should be softened to avoid ischemia due to the weight of the device and any other structures attached to it. The size of the intragastric soft building frame relative to the stomach can be varied based upon on the clinical application and the anatomy of the individual patient. The expanded dimension should be large enough to prevent passage through the pylorus.

The banana-shaped balloons of the building frame can be assembled in a number of different configurations.

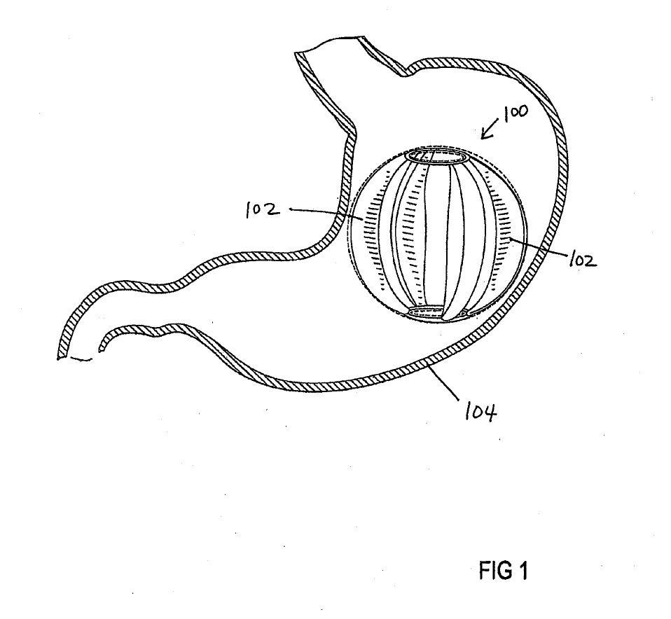

FIG. 1 shows the balloons 102 assembled in a convex-outward hourglass configuration 100. Depending on the curvature, size and spacing of the balloons 102, the assembled configuration 100 may look approximately like a football, a rugby ball or a soccer ball. This configuration of the building frame 100 will be good for long term use, such as to control flow of food and liquids through the stomach 104 because the rounded sides and ends will not place undue stress on the stomach walls. Optionally, the intragastric soft building frame 100 may be constructed with a membrane connecting the assembled balloons. The extent and location of the membrane covering the building frame 100, as well as the size and spacing of the balloons 102, will control the resistance to flow through the stomach 104.

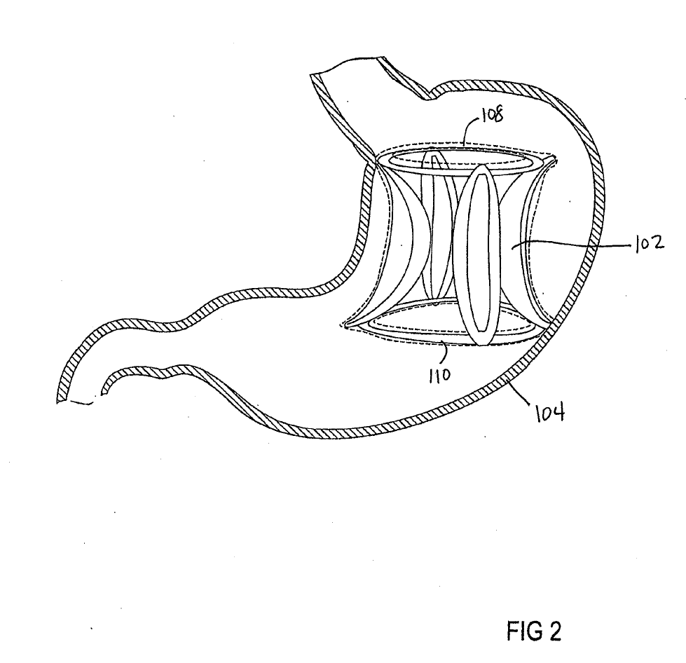

FIG. 2 shows the balloons 102 assembled in a concave-outward hourglass configuration 106. In this configuration 106, the building frame 106 will be stable and less likely to rotate within the stomach 104. Optionally, the intragastric soft building frame 106 may be constructed with a membrane connecting the balloons 102 around the outside, at the top 108, at the bottom 110 and/or at some intermediate portion. A membrane across the top 108 will make the building frame 106 useful as an intraoperative tool rest, whereas a membrane across the bottom 110 will make it more like a bucket for holding tools and components intraoperatively. When used to control flow of food and liquids through the stomach 104, the extent and location of the membrane covering the building frame 106, as well as the size and spacing of the balloons 102, will control the resistance to flow.

The inflatable balloons can be made of silicone, PU, PE, polyolefin, PET or other polymeric material. Materials such as PE and PET could be advantageous as they can be configured to have less distention, deflection and/or deformation and thereby provide improved mechanical support. Mechanical enhancements such as ribs, folds or reinforcing materials such as nylon or Kevlar fibers can also be included to enhance mechanical support. Balloon devices would preferably be inflated in place and would include inflating and/or deflating means. If the inflating/deflating means were removable, a reversible coupling means and/or a valve or inflation port sealing means could also be included. If used to support devices e.g. endoscopic sewing devices, mechanical coupling can optionally be included to interface with devices using the intragastric support. Examples of such couplings include U-shaped channels, rings, hooks, snaps and other means known in the art.

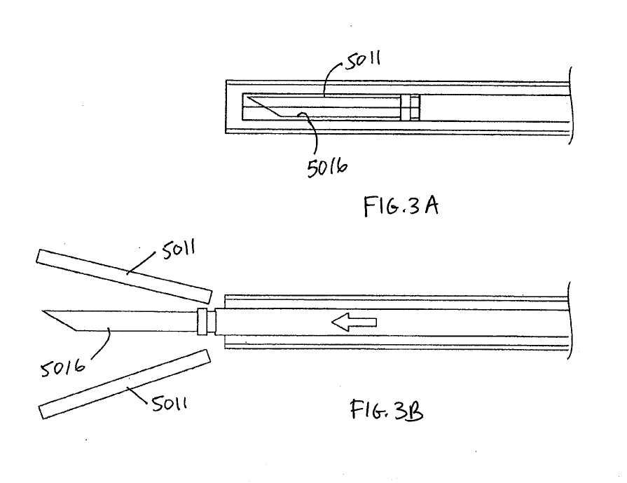

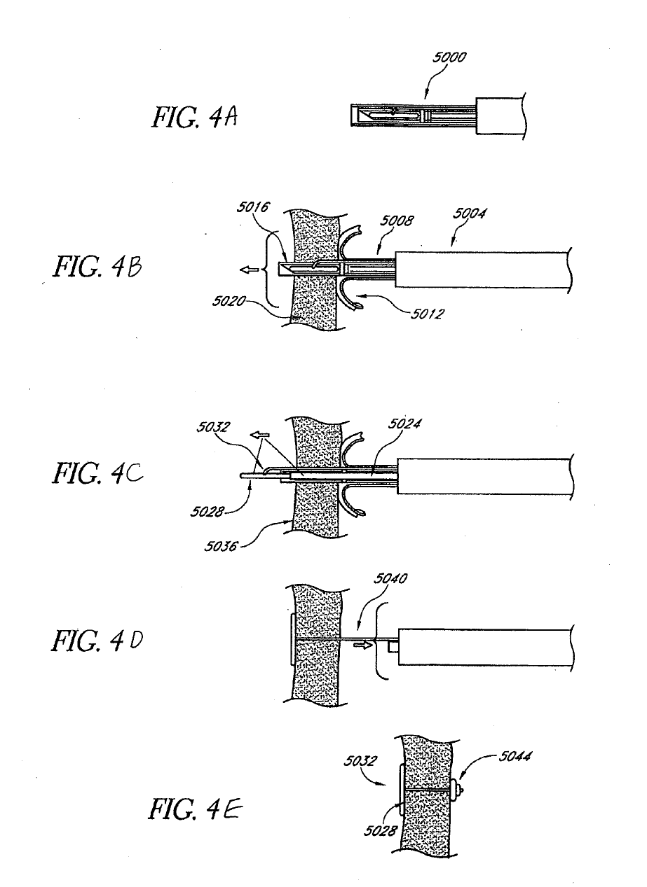

Devices and methods are described for sewing through the biopsy channel of a conventional endoscope, as shown in FIGS. 3-4. All the stages of sewing, cutting thread and tying knots or thread locking can be accomplished through a biopsy channel, optionally 2.8 mm or larger. The method does not require the use of endoscopic ultrasound (EUS), although it could be used with ultrasound real-time imaging to advantage to sew into specific organs or tissue depth.

Some endoscopic sewing methods use suction to control the depth of needle penetration into tissue. These include the BARD Endocinch and the Wilson Cook SewRight. These methods have two disadvantages. They increase the overall diameter of the endoscope from 11 mm to 15-18 mm depending on the size of the overtube or sewing capsule head. This makes the procedure uncomfortable for the patients and the procedure must be done under heavy sedation or general anesthesia. The other difficulty is that, when suction is applied to a cavity, the subsequent depth of gastric muscle penetrated by the needle is variable. This is in part due to the variable loose attachment of the mucosa and submucosa to the muscle and in part due to some variation in thickness of tissue. Another issue is that the tissue may be sucked into the cavity as two adjacent folds and the needle may run completely or partly between the folds thus failing to penetrate as deeply as is desirable. This seems to be due to large variations in stomach wall thickness, which is confirmed by measurements made of the stomach wall thickness in patients having resections for bleeding gastric ulcers (published in Gastroenterology in 1986). These measurements however were all performed on the wall adjacent to the ulcer, which was the point of interest for the study. Recent measurements of wall thickness with EUS at live surgery suggest that there may not be as much variation in wall thickness in healthy tissue. Nonetheless, wall thickness becomes a significant factor when it is important to sew to the correct depth using flexible endoscopy without knowledge of the gastric wall thickness. Pushing a needle into tissue tends to compress the mucosa and submucosa against the muscle, while suctioning the mucosa into a cavity tends to expand the distance to the serosa. Depending on the outer diameter of the needle and its sharpness and coefficient of friction, there may be some drag as the needle penetrates the tissue, which may increase the distance the needle must travel to penetrate to the serosa. The needle bevel is an important factor in the force required to push through tissue and will also influence the distance the T member of a T-tag fastener delivered through the needle must travel to reach its target. The distance of travel of the pushing rod may also need to be varied if the rod is to be used with the endoscope in both straight and extreme flexion configurations. The sewing method could be used with a T-tag fastener and suture as described herein. New knotting mechanisms and new ways of cutting thread are also disclosed. All of these can be deployed through a 2.8 mm diameter channel of a conventional gastroscope and do not require that the instrument be removed to tie knots or place extra stitches.

One goal of aspects of this invention is to develop new devices and methods for sewing during flexible endoscopy using sutures or fasteners, such as T-tag fasteners. The device includes a needle that can be pushed through tissue. There is an adjustable stop that allows penetration to a predetermined depth. The needle is short and is attached to a flexible shaft in order to allow the needle to be used in a flexed endoscope without restricting the bending radius of the scope, which is important, for example, for use at the cardio-esophageal junction. A method for expanding the stop mechanism is disclosed.

The needle 5016, shown in FIGS. 3A-3B, needs to be either short enough or flexible enough, to pass through the angulated entry of the biopsy channel just beyond the port below the hand controls of the flexible endoscope. In some embodiments, the needle 5016, for flexibility, can be formed of very thin stainless steel, NiTi, or a polymer. The needle 5016 may be sufficiently flexible, in some desirable embodiments, to be used without reducing the bending section at the tip. This differentiates it from the available EUS needles, which are too stiff to be used in a conventional flexible endoscope with more than about 30 degrees of bend. A high degree of flexibility is desirable for placing stitches under the cardio-esophageal junction, for example for treating GERD. If a rigid needle 5016 (made of e.g. stainless steel) is used, the length of the needle 5016 will preferably be about 1 cm or shorter. A very short needle 5016 could be made thicker than available EUS needles without compromising the ability to negotiate bends. The diameter of the needle 5016 is preferably about 18-20 gauge. The needle 5016 can preferably be soldered, welded or otherwise attached to a structure to transmit axial force. For example, the needle 5016 can be mounted on a braided or wire-wound, hollow catheter with a PTFE or other low friction coating. Alternatively, the needle 5016 can be mounted on a suitable plastic catheter or thin-walled metal tube. The needle catheter 5008 length must be sufficient to pass through the endoscope biopsy channel and connect to a handle with enough additional working length to reach the target tissue and carry out the sewing method as described herein.

The needle 5016 can be sheathed in order to protect the biopsy channel as the needle 5016 passes through the scope. One embodiment, shown in FIGS. 3A-3B, would use a short, disposable needle sheath 5011 that is ejected as soon as the needle 5016 reaches beyond the tip of the flexible endoscope. Another embodiment, shown in FIGS. 4A-4E, would use a split protective needle sheath 5012 with an innate springiness that would spring open as the needle 5016 moves beyond the tip of the flexible endoscope 5004. The opened split protective needle sheath 5012 would also act as a stop to control the depth of needle 5016 penetration into the tissue. The protective sheath 5012 could be metal, such as stainless steel or NiTi, or puncture resistant plastic, such as PE, PU, Nylon, and other similar materials. The sheath's 5012 functionality as a depth stop would not be affected by flexure of the endoscope. The split protective needle sheath 5012 would close automatically as the needle 5016 is withdrawn into the biopsy channel of the flexible endoscope 5004. Other embodiments comprising a distal rather than proximal depth stop are also contemplated as such distal depth stops can be advantageous because they are not affected by flexure of the scope 5004.

The device 5000 can preferably include a release mechanism for the T member 5028 and suture 5040 of a T-tag fastener 5032. A highly flexible wire push rod 5024, such as one formed of NiTi or stainless steel, could be used to eject the T member 5028 of the T-tag fastener 5032 from the distal end of the needle 5016 after it has penetrated the tissue 5020 to a predetermined depth. Hydraulic release of the T member 5028 would be another option. Alternatively, the T member 5028 of the T-tag fastener 5032 could be mounted on the end of the catheter 5008 to act as a needle 5016 for penetrating the tissue 5020. In this embodiment, the T member 5028 can include a penetrating point at its distal end. In another embodiment, the catheter 5008 could act as the pushing rod 5024 or a coaxial pushing rod 5024 could be used to separate the T member 5028 from the catheter 5008. The suture 5040 of the T-tag fastener 5032 could pass through the hollow catheter 5008 or outside of it.

The handle (not shown), which is connected to the needle catheter 5008, is preferably configured to provide precise control over the movement of the needle 5016 and the pushing rod 5024 or T-tag ejector to carry out the method as described below.

The suture 5040 of the T-tag fastener 5032 can be tied using conventional methods or the T-tag fastener 5032 may optionally include a suture locking mechanism 5044 as is known in the art.

By way of example, the sewing method is described below using the embodiment of the sewing device shown in FIGS. 4A-4E.

Method Steps:

The flexible endoscope 5004 is maneuvered to the target tissue.

The needle catheter 5008 is advanced through the biopsy channel of the scope 5004.

The split protective needle sheath 5012 opens as the needle 5016 emerges from the tip of the scope 5004.

The needle 5016 is plunged into the gastric tissue 5020 to a depth of 2-3 mm, with the open protective needle sheath 5012 acting as a stop to control the depth of needle 5016 penetration.

The pusher 5024 is advanced to eject the T member 5028 of the T-tag fastener 5032 from the distal end of the needle 5016 just beyond the serosal surface 5036.

The needle catheter 5008 is withdrawn into the biopsy channel of the scope 5004 and the split protective needle sheath 5012 closes.

The suture 5040 is secured by tying or by pushing a suture lock 5044 onto the suture 5040.

Optionally, the device may be configured to perform the sewing, locking and cutting of the suture in a single action. If the suture is passed through an open locking mechanism over the needle, the suture could be locked by pushing the catheter, sheath and lock forward.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

What is claimed is:

1. A method for deploying a tissue anchor through a channel in an endoscope and across a tissue wall, comprising:
providing a tubular body, having a sharpened distal end; a tissue attachment structure within the tubular body, the tissue attachment structure comprising a tension element and at least one retention surface, wherein the retention surface comprises a proximal surface of a serosal anchor; and a removable sheath surrounding at least the sharpened distal end, for isolating the sharpened distal end from a wall of the channel;
advancing the tubular body through a single, non-plicated tissue wall of the gastrointestinal tract;
ejecting the tissue attachment structure out of the tubular body such that the retention surface rests against a serosal surface of the tissue wall, wherein the retention surface transforms from a first, transversely reduced profile to a second, transversely enlarged profile upon being advanced distally out of the tubular body; and
withdrawing the tubular body proximally across the tissue wall, wherein the tension element spans the single non-plicated tissue wall after withdrawal of the tubular body.

2. The method of claim 1, wherein the serosal anchor comprises a T-tag.

3. The method of claim 1, wherein the tension element comprises suture.

4. The method of claim 1, wherein the tubular body comprises a needle.

5. The method of claim 1, wherein the at least one retention surface comprises a first retention surface extending in a first generally transverse direction with respect to a longitudinal axis of the tension element and a second retention surface extending in a second generally transverse direction with respect to the longitudinal axis of the tension element after passing transmurally through the wall.

6. The method of claim 1, wherein the tubular body is flexible.

7. The method of claim 1, further comprising the step of passing the tubular body through the channel of the endoscope prior to the advancing the tubular body step.

8. The method of claim 1, wherein the tubular body has a diameter of between about 18-20 gauge.
